(19) United States
(12) Patent Application Publication
(10) Pub. No.: US 2024/0294925 A1
(43) Pub. Date: Sep. 5, 2024

(54) Method of Treatment for Auditory Dysfunction

(71) Applicant: University of South Florida, Tampa, FL (US)

(72) Inventors: Subhra MOHAPATRA, Tampa, FL (US); Shyam MOHAPATRA, Tampa, FL (US); Courtney PENN, Tampa, FL (US)

(21) Appl. No.: 18/594,910

(22) Filed: Mar. 4, 2024

Related U.S. Application Data
(60) Provisional application No. 63/488,057, filed on Mar. 2, 2023.

(51) Int. Cl. C12N 15/113; A61K 35/28; A61K 45/06; A61K 47/69; A61P 27/16

(52) U.S. Cl. CPC C12N 15/1138; A61K 35/28; A61K 45/06; A61K 47/6929; A61P 27/16; C12N 2310/122; C12N 2310/14

(57) ABSTRACT

Methods are disclosed for auditory dysfunction by administration of a therapeutic agent and stem cells. The therapeutic agent is chosen from disclosed agents that target the CCL20-CCR6 axis. Further disclosed are treatment protocols of said therapeutic agent and stem cells. The method of treatment is disclosed to be treatment following a traumatic brain injury.

### CROSS REFERENCE TO RELATED APPLICATIONS

This patent application claims priority to, and the benefit of, U.S. provisional application, U.S. 63/488,057, filed on Mar. 2, 2023, which is hereby incorporated by reference herein in its entirety.

### STATEMENT OF GOVERNMENT SUPPORT

This invention was made with government support under Grant No. BX005757 awarded by the Department of Veteran Affairs. The Government has certain rights in the invention.

### BACKGROUND

Traumatic brain injury (TBI) affects over 2.5 million people annually in the US (Roup et al., 2020). TBI is a major cause of death and disability, and it contributes to at least 30% of injury-related deaths in the US (Faul et al., 2010). It is estimated that 1-2% of the population have one or more disabilities related to TBI (Kozin et al., 2021; Popescu et al., 2015). Even mild TBI, often called a concussion, can cause symptoms lasting for weeks to months after injury (Roup et al., 2020). This is especially true if someone experiences multiple TBIs (repetitive TBI, rTBI 1) over the course of their lifetime. This is a growing concern for amateur and professional athletes, and military veterans, who are at a higher risk of experiencing rTBI during their active-duty period. The damage caused by TBI is often divided into two phases. The primary injury phase results from direct mechanical damage to the brain. The secondary injury phase is caused by cellular and molecular changes occurring after injury that further exacerbate the initial damage. Mechanisms of secondary damage include the generation of free radicals, excitotoxicity, disruption of the blood-brain barrier, hypoxia, and inflammation (Kaur and Sharma, 2018). Many complications arising from TBI can be attributed to secondary injury mechanisms. Hence, mitigation of secondary injury damage has been a major goal of TBI research.

Post-concussive symptoms of TBI vary widely depending upon the location, severity, and method of injury. These can broadly be classified as physical, behavioral, sensory, or cognitive in nature. In the most extreme cases, rTBI can lead to chronic neurodegeneration and eventually symptoms of dementia, often referred to as chronic traumatic encephalopathy (CTE) (Blennow et al., 2016). Not all individuals who experience multiple TBIs will go on to develop CTE. Anxiety and depression are commonly reported postconcussive behavioral symptoms of TBI. An estimated 15-30% of patients report symptoms of anxiety after experiencing one or more mild TBIs, and self-reported anxiety levels have been associated with adverse health outcomes (Albayram et al., 2020; Lamontagne et al., 2022; Scholten et al., 2016; Stern et al., 2013; Terpstra et al., 2017). Depression has also been associated with other co-morbidities (Barker-Collo et al., 2015; Bombardier et al., 2016; Moreno-Lopez et al., 2016). Approximately 25-50% of TBI patients are diagnosed with major depression within 1 year of injury, a rate much higher than is seen in the general population (Scholten et al., 2016). Depression and suicide are also common in rTBI populations, and major depression has been suggested as a defining clinical feature of CTE (Guskiewicz et al., 2007; Kerr et al., 2012; Lehman et al., 2016; Webner and Iverson, 2016).

One of the most common post-concussive symptoms, and by far the most common sensory symptom, is hearing loss (Gombay and Andrews, 2021; Lew et al., 2011; Swan et al., 2018). Hearing loss after TBI can occur through damage to the inner ear, spinal cord, or central auditory system, including the auditory cortex. Damage to both hair cells and spiral ganglion has been observed in TBI patients (Ishai et al., 2018; Mao et al., 2021; Uchiyama et al., 2021). Hearing deficits are estimated to be 2-16 times more likely to develop in individuals with a history of TBI (Kozin et al., 2021; Lindquist et al., 2020; Shangkuan et al., 2017). Hearing loss has been reported in many studies of populations at risk for experiencing rTBI (Henry et al., 2021; Roup et al., 2020; Smith et al., 2020). A 2011 study of athletes with a history of TBI reported poorer auditory processing ability compared to non-concussed peers (Turgeon et al., 2011). The prevalence of auditory problems in veterans with a history of rTBI is higher compared to veterans without a history of rTBI (Henry et al., 2021; Moring et al., 2018; Roup et al., 2020; Swan et al., 2018). Tinnitus and hearing loss are the most common service-connected disabilities recorded by the Department of Veterans Affairs, costing billions of dollars each year (Henry et al., 2021; Moring et al., 2018). Although some cases of auditory dysfunction can be attributed to a direct impact to the ear or auditory pathway, auditory dysfunction can occur in the absence of this. Studies estimate that up to 58% of TBI cases without temporal bone fracture (TBF) result in a clinically relevant change in the hearing of at least 10-15 dB and 8% result in profound hearing loss or deafness (Lindquist et al., 2020). The mechanisms by which hearing loss occurs in these cases are not well understood; however, it is suggested to be the result of secondary injury mechanisms (Blennow et al., 2016; Lindquist et al., 2020). Treatment of hearing loss after TBI is limited to symptom management; no interventional therapies are available to mitigate secondary damage to the auditory system after TBI, and no FDA-approved drugs are available.

Neuroinflammation is a major cause of secondary injury spread after TBI (Burda et al., 2016; Donat et al., 2017; Karve et al., 2016). We have previously reported systemic changes in chemokine expression and increased activation of glial cells in the brain and retina following rTBI in a closed-head controlled cortical impact (CCI) model (Das et al., 2019; Mayilsamy et al., 2020). Although our rTBI model consists of mild impact to the parietal cortex, we have observed the eventual spread of inflammation and neurodegeneration to multiple other areas of the brain including the hippocampus and corpus callosum. The hippocampus is an important site of learning, memory, and emotional regulation; damage to hippocampal function has been linked to many post-concussive symptoms of TBI including memory loss, anxiety, depression, and post-traumatic stress disorder (Cha et al., 2016; Heller and Bagot, 2020; Logue et al., 2018; McKee et al., 2015; Schumacher et al., 2018). The corpus callosum is a large nerve tract enabling communication between the left and right hemispheres; it is a typical site of diffuse axonal injury following concussion (Armstrong et al., 2016; Johnson et al., 2013).

Our CCI rTBI rodent model recapitulates many symptoms experienced by TBI patients, especially those experiencing multiple TBI insults such as veterans and football players. We have observed changes in behavior, learning and memory, motor control, and retinal pathology (Das et al., 2019; Mayilsamy et al., 2020). However, the suitability of this model to investigate changes in auditory function or pathology within the auditory pathway remains unclear. Since our rTBI model exhibits secondary injury damage with enhanced neuroinflammation and neurodegeneration, we reasoned that it might provide an adequate model for an investigation into the mechanism of TBI-induced hearing loss. To this end, herein we characterize hearing loss in a CCI mouse model of rTBI and demonstrate the subsequent timeline of auditory dysfunction and pathology.

### SUMMARY

Disclosed herein are methods that CCL20-CCR6 is a target pathway involved in the loss of auditory function, loss of spiral ganglion, and neuroinflammation in the brain and cochlea as early as 14 days post-traumatic brain injury, which persists up to 60 days post-injury. Further, it is contemplated that reductions of CCL20 after rTBI may mitigate inflammation and neurodegeneration of spiral ganglion at 14 days post-injury. Hence inhibitors targeting this pathway can be therapeutic targets against auditory illness following TBI.

A mouse model of rTBI was established in order to determine a timeline of auditory pathology following multiple mild injuries. Mice were subject to controlled cortical impact at the skull midline once every 48 h, for a total of five hits. Auditory function was assessed via the auditory brainstem response (ABR) at various timepoints post injury. Brain and cochleae were collected to establish a timeline of cellular pathology.

Increased ABR thresholds and decreased (ABR) P1 amplitudes were observed in rTBI vs sham animals at 14 days post-impact (dpi). This effect persisted for up to 60 days (dpi). Auditory temporal processing was impaired beginning at 30 dpi. Spiral ganglion degeneration was evident at 14 dpi. No loss of hair cells was detected at this time, suggesting that neuronal loss is one of the earliest notable events in hearing loss caused by this type of rTBI.

It was observed that rTBI results in chronic auditory dysfunction via damage to the spiral ganglion which occurs in the absence of any reduction in hair cell number. This suggests early neuronal damage that may be caused by systemic mechanisms similar to those leading to the spread of neuronal death in the brain following TBI. This TBI-hearing loss model provides an important first step towards identifying therapeutic targets to attenuate damage to the auditory system following head injury.

In some aspects, a method of treatment for auditory dysfunction is disclosed. The method including: administering a therapeutic amount of a therapeutic agent to inhibit chemokine ligand 20 (CCL20) and/or chemokine receptor 6 (CCR6) one of a dendrimer nanoparticle complexed with at least one short hairpin RNA (shRNA)-encoding DNA plasmid of at least one dendriplex, or a PPARγ agonist; and administering a therapeutic amount of stem cells.

### BRIEF DESCRIPTION OF THE DRAWINGS

FIG. 1A shows a schematic of rTBI model and timeline.

FIG. 1B shows representative images of FluoroJade-C staining at 14-, 30-, and 60-days post injury.

FIGS. 1C-1E show impact site and auditory cortex and quantification of FluoroJade-C+(FJ+) cells at 14 days post-injury (FIG. 1C), 30 days post injury (FIG. 1D), and 60 days post injury (FIG. 1E).

FIG. 2A shows a representative heatmaps of open field test where shading indicates time spent in area.

FIGS. 2B-2D show quantification time spent in center area, number of entries into center area, and time spent in corner areas during open field test at 14 days post-injury (FIG. 2B), 30 days post injury (FIG. 2C), and 60 days post injury (FIG. 2D).

FIGS. 2E-2G show quantification of sucrose intake (%) by volume (% sucrose consumed/total liquid consumed) at 14 days post-injury (FIG. 2E), 30 days post injury (FIG. 2F), and 60 days post injury (FIG. 2G).

FIG. 2H shows two-way analysis of variance for sucrose preference test.

FIG. 3A shows representative images of brain sections stained for Iba-1 (microglia) and DAPI (cell nuclei) of rTBI induced microgliosis in multiple brain regions: auditory cortex (AC), corpus callosum (CC), impact site (parietal cortex, IS), and hippocampus (HC) (n=8 mice per group). White rectangles represent magnified areas (right).

FIGS. 3B-3D show quantification of Iba-1+cells in AC, CC, IS, and HC at 14 days post-injury (FIG. 3B), 30 days post injury (FIG. 3C), and 60 days post injury (FIG. 3D).

FIG. 3E shows student's t-test results from various brain regions (Iba1 and GFAP positive cells.

FIG. 4A shows representative images of brain sections stained for GFAP (astrocytes, green) and DAPI (blue, cell nuclei) of rTBI induces astrogliosis in multiple brain regions: auditory cortex (AC), corpus callosum (CC), impact site (parietal cortex, IS), and hippocampus (HC) (n=8 mice per group). White rectangles represent magnified areas (right).

FIGS. 4B-4D show quantification of GFAP+cells in AC, CC, IS, and HC at 14 days post-injury (FIG. 4B), 30 days post injury (FIG. 4C), and 60 days post injury (FIG. 4D).

FIGS. 5A-5J shows representative ABR traces of hearing impairment following rTBI (n=8 mice per group (FIG. 5A), ABR threshold from 3 kHz to 48 kHz stimulus frequencies and wideband noise (WBN) at 14 days post-injury (FIG. 5B), 30 days post injury (FIG. 5C), and 60 days post injury (FIG. 5D); ABR Peak I amplitude from 3 kHz to 48 kHz stimulus frequencies and WBN at 14 days post-injury (FIG. 5E), 30 days post injury (FIG. 5F), and 60 days post injury (FIG. 5G); ABR Peak IV amplitude from 3 kHz to 48 kHz stimulus frequencies and WBN at 14 days post-injury (FIG. 5H), 30 days post injury (FIG. 5I), and 60 days post injury (FIG. 5J).

FIG. 5K shows two-way analysis of variance for ABR measurements.

FIGS. 6A-6F show ABR Gap-in-Noise (ABR GIN) recovery ratio for stimulus gaps of 1-64 milliseconds at 14 days postinjury (FIG. 6A), 30 days post injury (FIG. 6B), and 60 days post injury (FIG. 6C); ABR GIN minimum gap threshold (MGT) at 14 days post-injury (FIG. 6D), 30 days post injury (FIG. 6E), and 60 days post injury (FIG. 6F.

FIG. 6G shows two-way analysis of variance for ABR GIN recovery ratios.

FIG. 7A shows representative images of cochlear cryosections stained with hematoxylin and eosin (H&E).

FIG. 7B-7D show quantification of spiral ganglion cell count from cochlear cryosections stained with H&E at 14 (FIG. 7B), 30 (FIG. 7C), and 60 (FIG. 7D).

FIG. 7E shows representative images of cochlear cryosections stained for neurofilament (NF-H) and DAPI (cell nuclei) of basal, middle, and apical regions (scale bar=50 m).

FIG. 7F-7H show quantification of NF-H+spiral ganglion neurons from cochlear sections stained for NF-H and DAPI in basal (FIG. 7F), middle (FIG. 7G), and apical (FIG. 7H) regions.

FIG. 8A shows cochlear whole mounts stained for neurofilament (Scale bar=20 m) shows a loss of spiral ganglion dendrites and reduction of inner hair-cell size at 14 days post-injury (n=8 mice per group).

FIG. 8B shows quantification of missing hair cells in basal, middle, and apical cochlear whole mounts.

FIG. 8C shows quantification of dendrite counts from basal, middle, and apical cochlear whole mounts.

FIG. 8D shows representative images of cochlear cryosections stained for myosin VIIa (hair cells), NF-H, and DAPI.

FIG. 8E shows quantification of inner hair cell area from cochlear sections stained for myosin VIIa, NF-H, and DAPI.

FIG. 9 shows cochlear cryosections from 14 days post-injury (n=8).

FIG. 10A shows hematoxylin/eosin staining of cochlea from sham, rTBI, and rTBI mice treated with shRNA nanodendriplexes targeted to CCL20/CCR6 (shCombo) (n=3).

FIG. 10B shows quantification of spiral ganglion cells from rTBI and shCombo mice (n=3).

FIG. 11A shows Iba1 staining of auditory cortex from sham, rTBI, and shCombo mice (n=3).

FIG. 11B shows quantification of Iba1+cells from rTBI, and shCombo mice (n=3).

### DETAILED SPECIFICATION

To facilitate an understanding of the principles and features of various embodiments of the present invention, they are explained hereinafter with reference to their implementation in illustrative embodiments.

In the following detailed description, reference is made to the accompanying drawings, which form a part hereof, and within which are shown by way of illustration specific embodiments by which the disclosure can be practiced. It is to be understood that other embodiments by which the disclosure can be practiced. It is to be understood that other embodiments can be utilized, and structural changes can be made without departing from the scope of the disclosure.

### General Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supersedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about.” It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. Where particular values are described in the application and claims, unless otherwise stated, the term “about” meaning within an acceptable error range for the particular value should be assumed. In some cases, the term “about” means+15%.

Concentrations, amounts, solubilities, and other numerical data can be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about one to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range, or the characteristics being described.

As used in the specification and claims, the singular form “a,” “an” and “the” include plural references unless the context clearly dictates otherwise. For example, the term “a nanoparticle” includes a plurality of nanoparticles, including mixtures thereof.

“Patient” is used to describe an animal, preferably a human, to whom treatment is administered, including prophylactic treatment with the compositions of the present disclosure.

The “therapeutically effective amount” for purposes herein is thus determined by such considerations as are known in the art. A therapeutically effective amount of other indicators are selected as appropriate measures by those skilled in the art. In accordance with the present disclosure, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

“Administration” or “administering” is used to describe the process in which a small molecule inhibitor. The composition can be administered in various ways including parenteral (referring to intravenous, intraarterial and other appropriate parenteral routes), intraocular, topically, orally, and percutaneously, among others.

Traumatic Brain Injury (TBI) is a major cause of death and disability worldwide. Although the majority of TBIs are classified as mild, repetitive mild injuries (rTBI) can lead to chronic post-concussive symptoms such as hearing loss. Treatment for TBI-induced hearing loss is limited to symptom management, and the secondary pathways that may affect or worsen damage to the auditory system are not well understood. We have previously shown that the inflammatory cytokine CCL20 is a major player in rTBI-induced pathology. We hypothesize that rTBI-induced auditory dysfunction may also be mediated by an inflammatory cascade involving CCL20.

A loss of auditory function, loss of spiral ganglion, and neuroinflammation were observed in the brain and cochlea. Results indicate that reduction of CCL20 after rTBI may mitigate inflammation and neurodegeneration of spiral ganglion.

It is disclosed that rTBI causes chronic hearing loss and neurodegeneration in mouse models, and it is contemplated that targeting CCL20 to attenuate rTBI-induced hearing loss is an effective treatment method.

Disclosed herein are methods of treatment for auditory dysfunction. It is contemplated that the cause of auditory dysfunction is a secondary injury from traumatic brain injury. It has been shown that C-C chemokine 20 (CCL20) plays a critical role in neurodegeneration and inflammation following TBI (Das et al., J Neuroinflammation 8:148, 2011). C-C chemokine receptor 6 (CCR6) is the only receptor that CCL20 interacts with. The methods disclosed herein therefore target the CCL20-CCR6 pathway as has been shown to effectively treat auditory dysfunction.

In some aspects, the method of treatment for auditory dysfunction includes: administering a therapeutic amount of a therapeutic agent to for down-regulating chemokine ligand 20 (CCL20) and/or chemokine receptor 6 (CCR6) one of a dendrimer nanoparticle complexed with at least one short hairpin RNA (shRNA)-encoding DNA plasmid of at least one dendriplex, or a PPARγ agonist; and administering a therapeutic amount of stem cells.

In some aspects, auditory dysfunction is a result of traumatic brain injury.

In some aspects, the therapeutic agent for down-regulating CCL20 and/or CCR6 includes one of a dendrimer nanoparticle complexed with at least one short hairpin RNA (shRNA)-encoding DNA plasmid of at least one dendriplex, or a peroxisome proliferator-activated receptor gamma (PPARγ) agonist.

In some aspects, the at least one shRNA-encoding DNA plasmid contains at least one shRNA encoding CCL20, CCR6 or a combination thereof.

In some aspects, the dendrimer nanoparticle is a polyamidoamine (PAMAM) dendrimer. The PAMAM dendrimer may further include a cyanine7 ligand.

In some aspects, the PPARγ agonist includes pioglitazone rosiglitazone, troglitazone, englitazone, balaglitazone, rivoglitazone, ciglitazone, lobeglitazone, or netoglitazone, honokiol, amorfrutin 1, amorfrutin B, and amorphastilbol, or a pharmaceutically acceptable salt thereof.

In some aspects, the PPARγ agonist includes two or more PPARγ agonists.

In some aspects, the stem cells are human mesenchymal stem cells.

In some aspects, the therapeutic agent for down-regulating CCL20 and/or CCR6 is administered before administering the stem cells.

In some aspects, the therapeutic agent for down-regulating CCL20 and/or CCR6 is administered concurrently with administering the stem cells.

In some aspects, the techniques described herein relate to a method, wherein the therapeutic agent for down-regulating CCL20 and/or CCR6 is administered after administering the stem cells.

In some aspects, the therapeutic agent for down-regulating CCL20 and/or CCR6 is administered intranasally.

In some aspects, the therapeutic agent for down-regulating CCL20 and/or CCR6 is administered orally.

In some aspects, the therapeutic agent for down-regulating CCL20 and/or CCR6 is administered intravenously.

In some aspects, the stem cells are administered intravenously.

In some aspects, the stem cells are administered intranasally.

In some aspects, the stem cells are administered intra-arterially.

In some aspects, the stem cells are administered intra-cranially.

The following examples are given for demonstrative purposes and are not intended to limit the scope of the disclosure.

### EXAMPLES

### Example 1: Early cochlear neurodegeneration in the absence of hair cell loss

Methods. All animal procedures were conducted in accordance with the NIH guidelines for the Care and Use of Laboratory Animals and approved by the Institutional Animal Care and Use Committee of the University of South Florida. 48, 6-8-week-old male C57BL/6 mice (Envigo) were housed in the vivarium on a 12 hour light/12 hour dark cycle with food and water available ad libitum. After rTBI induction, behavioral tests, and hearing tests, mice were deeply anesthetized with euthasol (150 μg/ml, given by intraperitoneal injection), and perfused with PBS and 4% paraformaldehyde prior to tissue collection. Mice were split into groups of 16 (8 sham, 8 rTBI) per timepoint. One mouse in the 60 days post-injury (dpi 1) rTBI subject group was lost before completion of behavioral tests; data is only shown for the remaining 7 rTBI mice in that group.

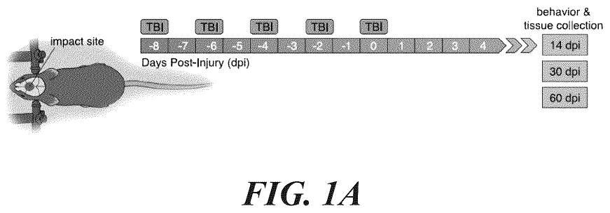

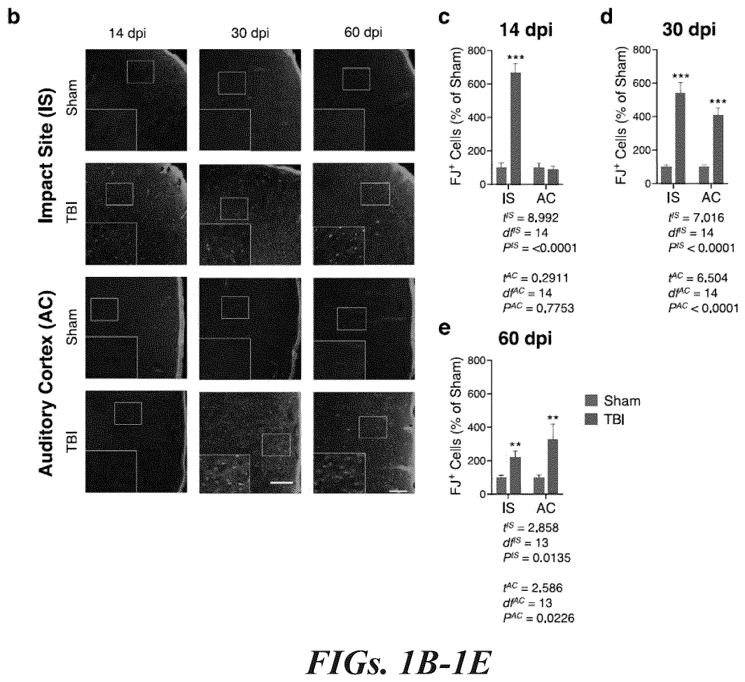

Repetitive traumatic brain injury (rTBI) was performed as previously described (Das et al., 2019; Mayilsamy et al., 2020). Mice were kept under anesthesia with 2% isoflurane throughout the closed head rTBI procedure. Hair was removed from the impact area prior to rTBI. The skull was not opened. Mice were fixed in a stereotactic device and subject to TBI via a controlled cortical impactor (Leica). The 5 mm diameter impactor tip was positioned at −0.8 mm from bregma on the midline. FIG. 1A shows an approximate location of the impact site. Impact was delivered at 5 m/s at a depth of 1.5 mm with 200 milliseconds dwell time. After injury, mice were allowed to recover on a heating pad and then returned to their home cage. TBI was performed every 48 h over the course of nine days for a total of 5 hits. Days post-injury (dpi) was measured with respect to the final (5th) TBI. Behavioral tests, hearing tests, and tissue collection were performed at 14, 30, and 60 dpi (see FIG. 1A).

Animals were subject to gentle handling by the experimenter for 3-5 days and introduced to the behavior room prior to the open field test. Mice were recorded exploring a 2′×2′ open field for 5 minutes with a 1-minute acclimation period in the presence of soft white-noise. Recording, tracking, analysis, and generation of heatmaps was done using ANY-Maze software.

FluoroJade-C staining was performed as previously described (Das et al., 2019; Mayilsamy et al., 2020). Coronal brain sections of 30 m were hydrated using 100%, 70%, and 30% EtOH followed by deionized water. Sections were oxidized in 0.06% KMnO₄ and then incubated with a 0.001% solution of FluoroJade-C in 0.1% acetic acid. FluoroJade-C was visualized using a Keyence fluorescence microscope (BZ-X710, Keyence America, IL, USA).

Animals were subject to gentle handling by the experimenter for 3-5 days prior to sucrose preference test. Mice were separated into individual cages and allowed to acclimate for 24 hours prior to sucrose introduction. After this acclimatization period, mice were given access to a standard water bottle as well as a water bottle containing 1% sucrose. The side of the cage containing sucrose was randomly assigned and switched every 12 hours to rule out differences in side-preference. Water bottles were weighed prior to being placed in the cage and subsequently weighed every 12 hours for a total of 5 days.

Immunohistochemistry was performed as previously described (Das et al., 2019; Mayilsamy et al., 2020). Coronal brain sections of 30 m or 5 m cochlear sections were subject to antigen retrieval using a citrate-based unmasking solution (Vector labs), blocked in 10% normal goat serum with 0.3% triton, and incubated with primary antibodies overnight. Sections were subsequently incubated with secondary fluorescence antibodies for 2 hours at room temperature and visualized using a Keyence fluorescence microscope (BZX710, Keyence America, IL, USA). Antibodies used in this study are as follows: Ionized calcium-binding adapter moleculel (Iba1-Rb, Wako 1:500), Glial fibrillary acidic protein (GFAP-Chk, Millipore 1:500), Neurofilament H (NF-H-Chk, Millipore 1:500), Myosin VIIa (MyoVII-Rb, Proteus 1:10 00), Alexa Fluor Rb-594 (Invitrogen, 1:1000), Alexa Fluor Chk-488 (Invitrogen, 1:1000).

Hearing tests were performed as previously described (Williamson et al., 2015). Briefly, mice were anesthetized with ketamine/xylazine (120/10 kg/mg body weight) via intraperitoneal injection. Mice were kept on a heating pad to maintain body temperature throughout recording. Auditory stimuli were presented in a soundproof booth through an electrostatic speaker (TDT EC1, Tucker Davis Tech., Alachua FL) connected to a coupler that was inserted directly into the ear canal. The TDT system was calibrated daily. Needle electrodes were inserted subcutaneously at the vertex, ipsilateral mastoid area, and posterior to the contralateral pinna. Auditory brainstem response (ABR) tonal stimuli ranged from 3 to 48 kHz, at 5 dB steps. A wideband noise (WBN 1) stimulus with bandwidth 0-48 kHz was also used. ABR Gap-In-Noise (ABR GIN 1) consisted of two WBN bursts with a silent gap of varying length inserted into the center. Responses were recorded using BioSig software (TDT).

Cochlear cryosections of 5 m were hydrated with graded ethanol, incubated with hematoxylin for 3 minutes, bluing agent 1 minute, and eosin 1 minute. Slides were dehydrated prior to cover-slipping and visualization on a Keyence light microscope (BZ-X710, Keyence America, IL, USA).

Cochlear whole mounts were prepared as described in a protocol published by Montgomery et al. detailing isolation of the organ of Corti as three intact cochlear turns (Montgomery and Cox, 2016). Whole mount preparation was performed via dissection in PBS after decalcification in 5% EDTA. The resulting cochlear turns were stained using immunohistochemistry as described above, then mounted on slides before visualization using an Olympus confocal laser scanning microscope (Olympus FV1200).

Generation of open field measurements was performed using ANY-Maze software. Cell counts of FluoroJade-C, H&E, and immune-stained brain/cochlear sections as well as inner hair cell area measurements were performed using ImageJ software. Numbers represent averages from at least five images per mouse. Hearing test measurements were performed using BioSig software (TDT). Statistics and graph preparation were performed using GraphPad Prism. Statistical tests are indicated in figure legends.

Results. TBI causes immediate cellular damage to the impact site that may spread over time due to secondary injury mechanisms. Neurodegeneration was previously reported at the injury site following rTBI as early as 7 dpi (Das et al., 2019; Mayilsamy et al., 2020). In order to establish a timeline of hearing pathology after rTBI, mice were subjected to either rTBI via CCI or sham (anesthesia only) treatment. Mice were given behavioral and hearing tests at 14, 30, and 60 dpi (per FIG. 1A). After behavioral and hearing tests, brain and cochlear tissues were collected.

To verify the successful administration of rTBI and assess neurodegeneration in the auditory cortex, coronal brain sections of sham and rTBI mice were stained with FluoroJade-C (see FIGS. 1B-1E), a fluorescent dye that specifically labels degenerating neurons. An increase in the number of FJC-positive cells were observed at the injury site (parietal cortex near midline) at 14, 30, and 60 dpi in rTBI mice. FJC-positive cells increased in the auditory cortex at later timepoints (30 and 60 dpi), but not at 14 dpi. This suggests neurodegeneration which begins at the injury site and spreads to the auditory cortex over time.

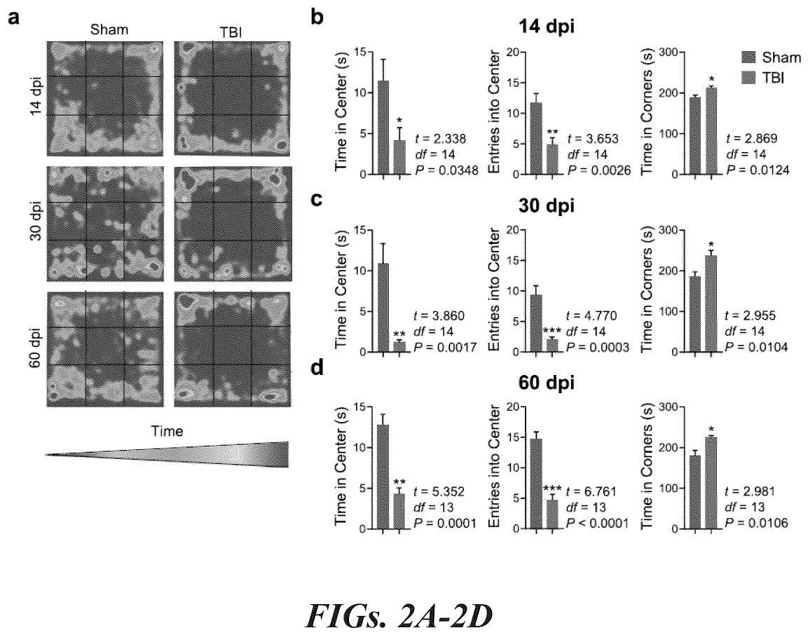

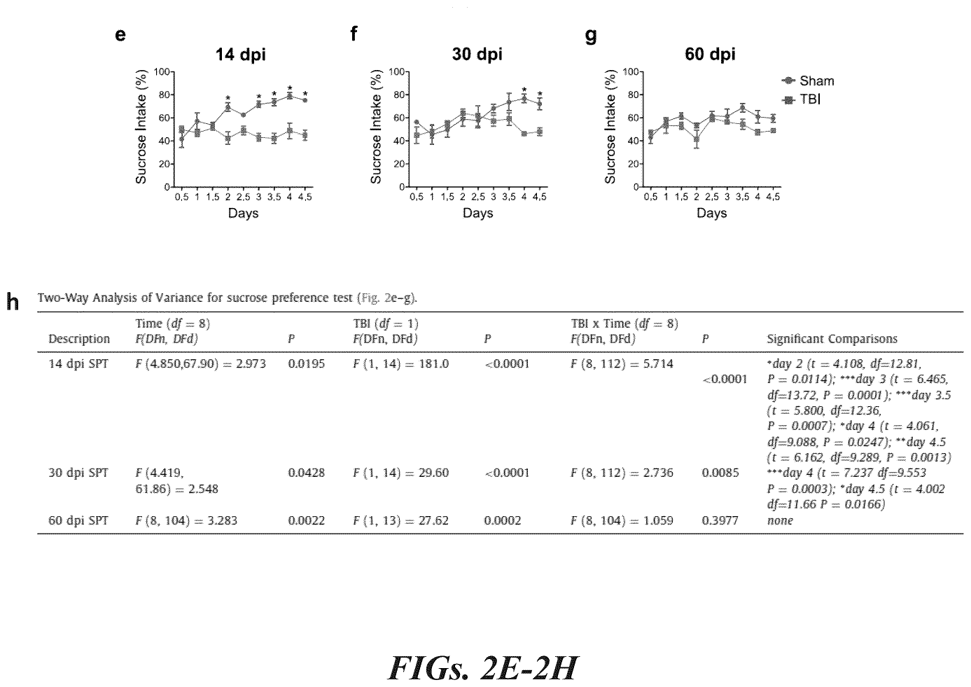

In order to determine whether the rTBI mouse model recapitulates symptoms of anxiety, sham and rTBI mice were subjected to an open field test at 14, 30, and 60 dpi (see FIGS. 2A-2D). Mice were placed into a walled enclosure and allowed to explore freely for 5 minutes. Reluctance to explore the center of an open enclosure has been linked to increased anxiety in a variety of rodent models (Seibenhener and Wooten, 2015). A significant reduction in time spent in the center area of the open field, a decrease in the number of entries into the center area, and increased time spent in corner areas at 14 dpi (see FIGS. 2A and 2B) were observed. This behavioral phenotype could also be observed at 30 dpi (FIGS. 2A and 2C) and 60 dpi (FIGS. 2A and 2D) suggesting chronic anxiety following rTBI in this model.

To assess depressive behaviors in the mouse model, sham and rTBI mice were subjected to a sucrose preference test at 14, 30, and 60 dpi (FIG. 2E-2H). Mice were separated and allowed to choose between drinking from a standard water bottle or from a water bottle containing 1% sucrose. The locations of the two bottles were swapped each day. Sucrose intake (fraction of total liquid consumption comprising 1% sucrose solution) was measured every 12 hours over the course of 5 days. During this time, sucrose intake often increases as the mouse learns to seek out the sweeter water. A lack of preference for sucrose in mice has been interpreted as a sign of anhedonia, a core symptom of depression (Liu et al., 2018). Decreased sucrose intake in rTBI mice compared to sham at 14 and 30 dpi (see FIGS. 2E and 2F) was observed. At 60 dpi this appears to be less pronounced (see FIG. 2G), but a significant major effect of rTBI on sucrose intake remained (2-way ANOVA). This indicates that rTBI mice experienced depressive behavioral symptoms which become less severe over the course of two months after injury.

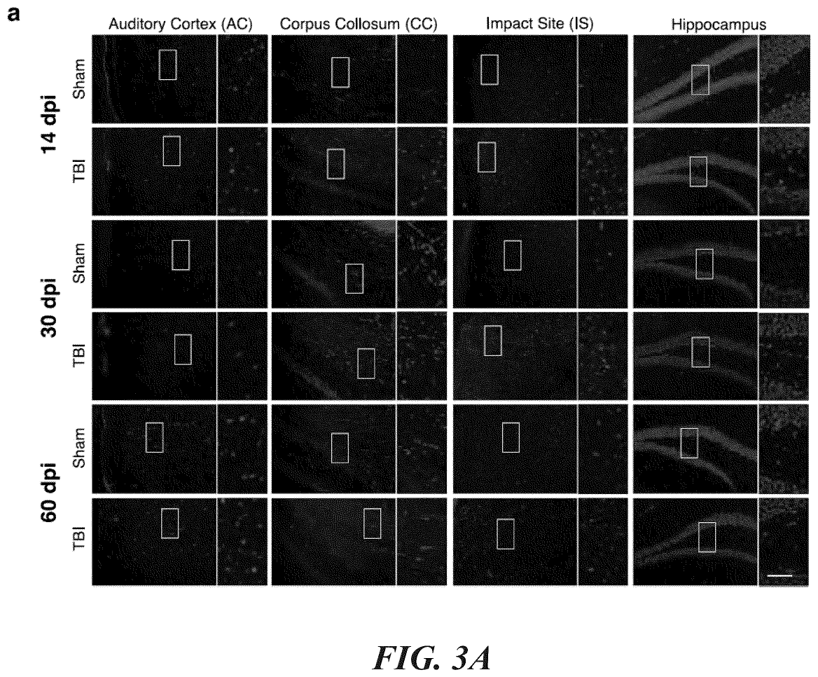

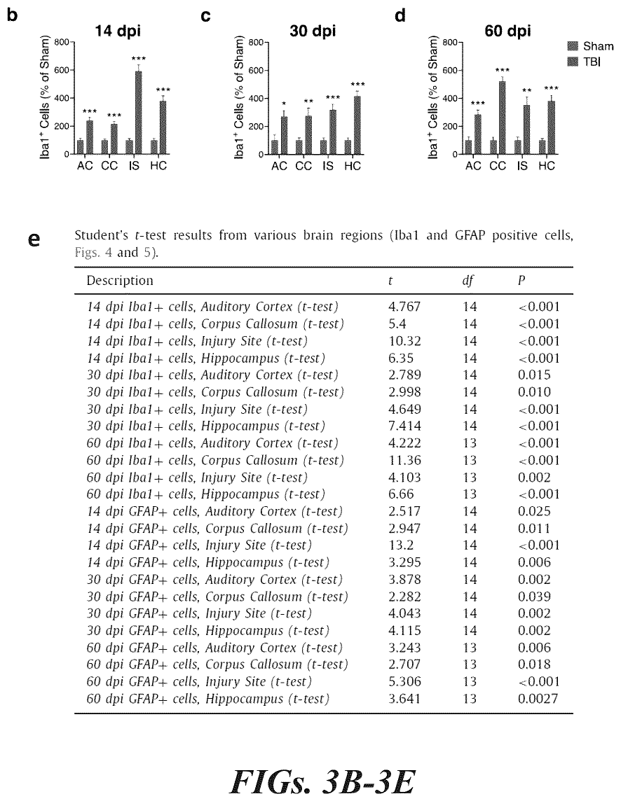

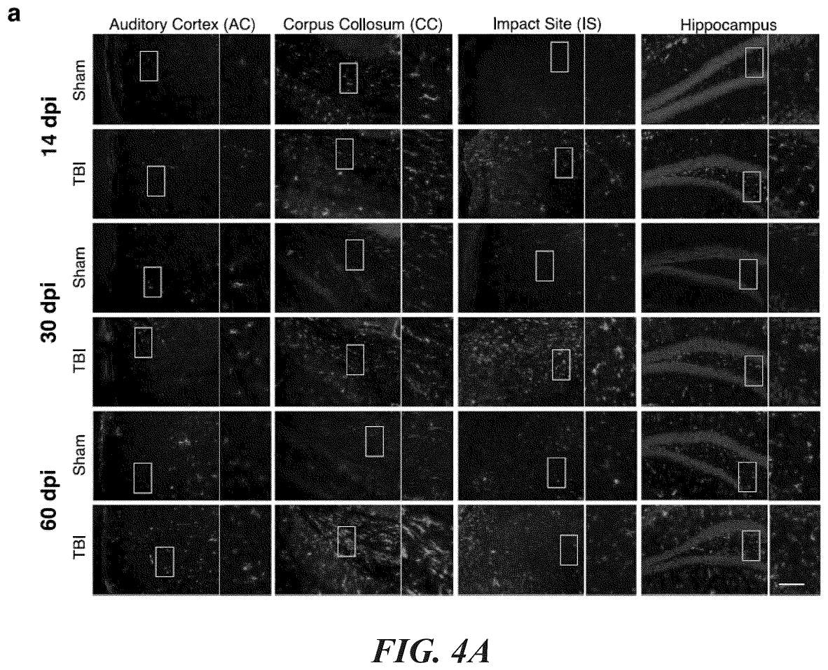

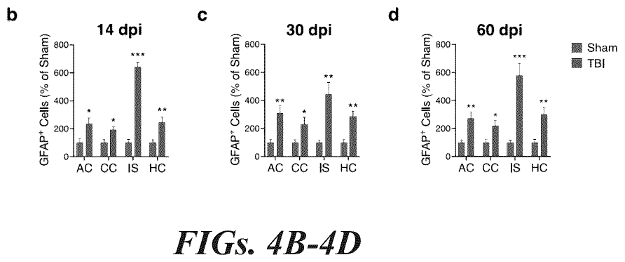

In order to observe changes in glial activation following rTBI, immunostaining of sham and rTBI brain sections was performed. The analysis was focused on four brain regions relevant to the study: the impact site (parietal cortex, midline), auditory cortex, hippocampus, and corpus callosum. Brain sections of sham and rTBI mice were stained for Iba1, which is upregulated during microglial activation. Rather than localized microgliosis at the injury site, widespread activation of microglia was observed throughout multiple brain regions at 14, 30, and 60 days following rTBI (see FIGS. 3A-3E). The largest increase in microgliosis appeared to be concentrated at the injury site and hippocampus at 14 dpi (see FIG. 3B), and more evenly spread to other regions at further timepoints (see FIGS. 3C-3D). In addition, brain sections of sham and rTBI were stained for GFAP, a marker of astrocyte activity. Similar to the microgliosis observed after rTBI, astrogliosis was widespread rather than localized to the injury site at all three timepoints (14, 30, and 60 dpi) (see FIG. 4A-4D and FIG. 3E). However, activation of astrocytes was more concentrated around the injury site in comparison to microglial activation (see FIGS. 4B-4D).

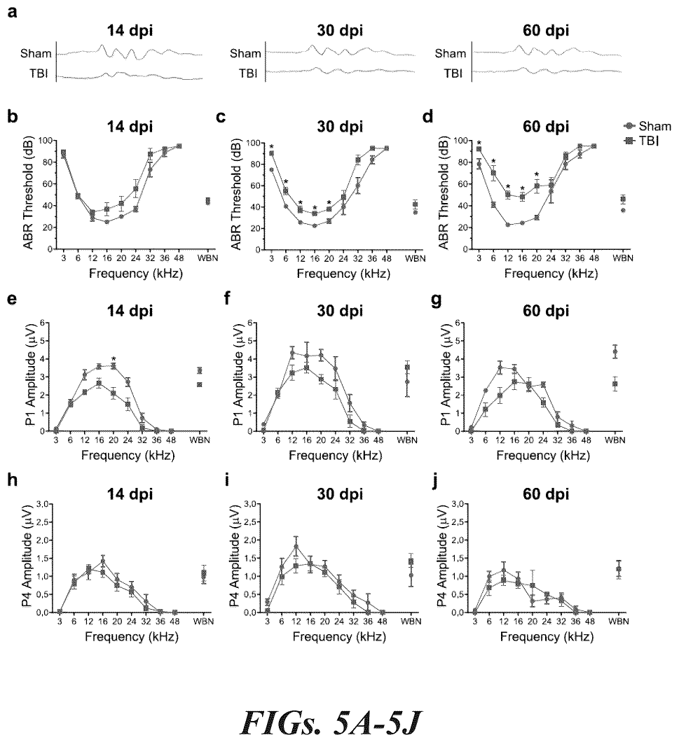

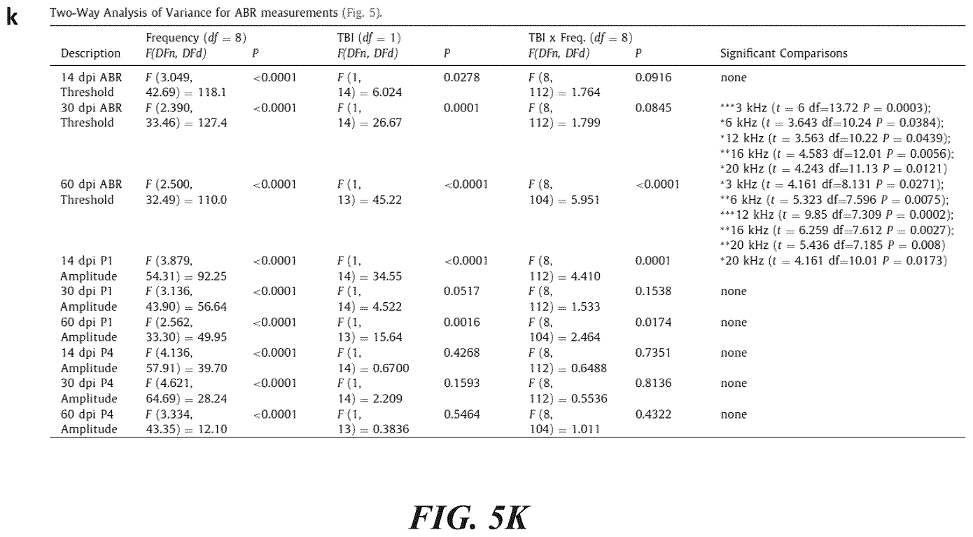

Behavioral deficits and neurodegeneration in the brain and eye following rTBI in mouse models have previously been reported; however, the effects of rTBI on the auditory system in this model were previously unexplored. Therefore, the auditory brainstem response (ABR) was measured, and it evoked potentials consisting of a sequence of five peaks (P1-P5) via scalp electrodes, in sham and rTBI mice at 14, 30, and 60 dpi (see FIGS. 5A-5K). rTBI increased ABR thresholds at all timepoints (see FIGS. 5B-5D), particularly in midrange frequencies at 30 and 60 dpi. This is accompanied by a reduction in P1 amplitude following rTBI (see FIGS. 5E-5G). P1 is believed to be generated by spiral ganglion neurons and is correlated to the integrity of cochlear synapses and nerve fibers (McClaskey et al., 2020; Paquette et al., 2016; Rattay and Danner, 2014). No changes in P4 amplitude (see FIGS. 5H-5J) were induced by rTBI. This suggests possible damage to the auditory pathway at the level of the inner ear and cochlear nerve in rTBI mice. Significant alterations in the latency of P1 or P4 were not observed after rTBI.

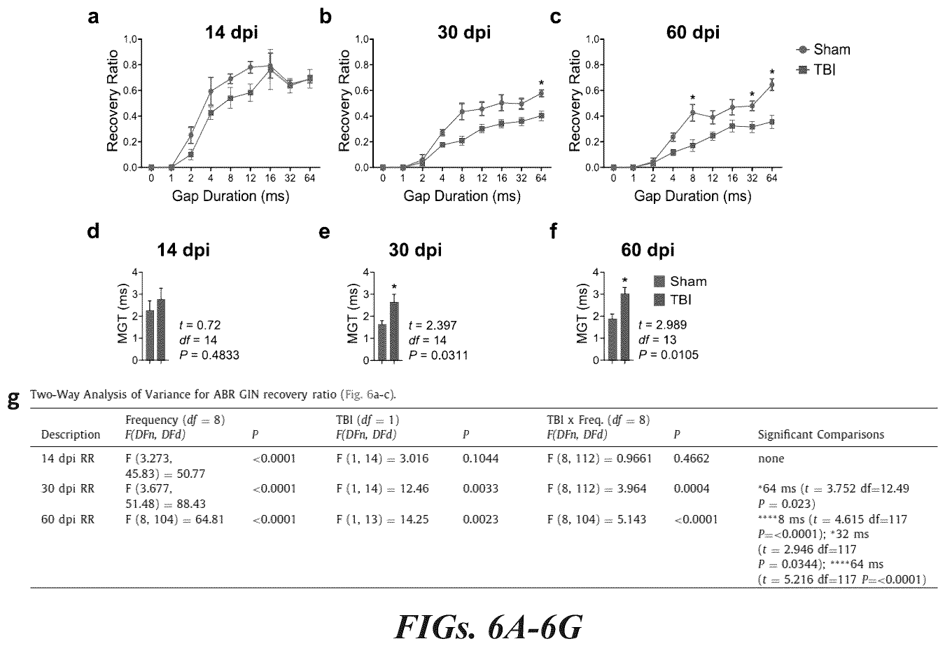

In order to measure auditory temporal resolution after injury, sham and rTBI mice underwent ABR Gap-In-Noise (ABR GIN) hearing tests (Williamson et al., 2015) (see FIGS. 6A-6G). During this procedure, two separate noise stimuli were given and the temporal gap between them was varied from 0 to 64 milliseconds. As the time between the two sounds increased, the P1 recovery ratio (ratio of second P1 ABR amplitude to first P1 ABR amplitude) approached unity. Deficiency in P1 recovery ratio was observed beginning at 30 dpi and persisting until 60 dpi (see FIGS. 6B-6C). An increase in minimum gap threshold (MGT), the minimum time needed between stimuli to evoke a second ABR response, was also observed at the same timepoints post-injury (see FIGS. 6E-6F).

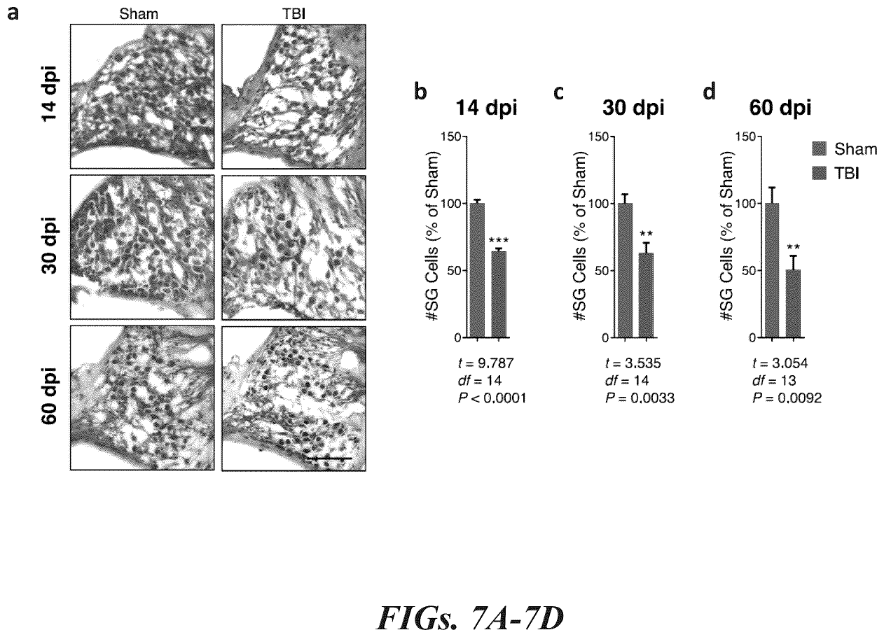

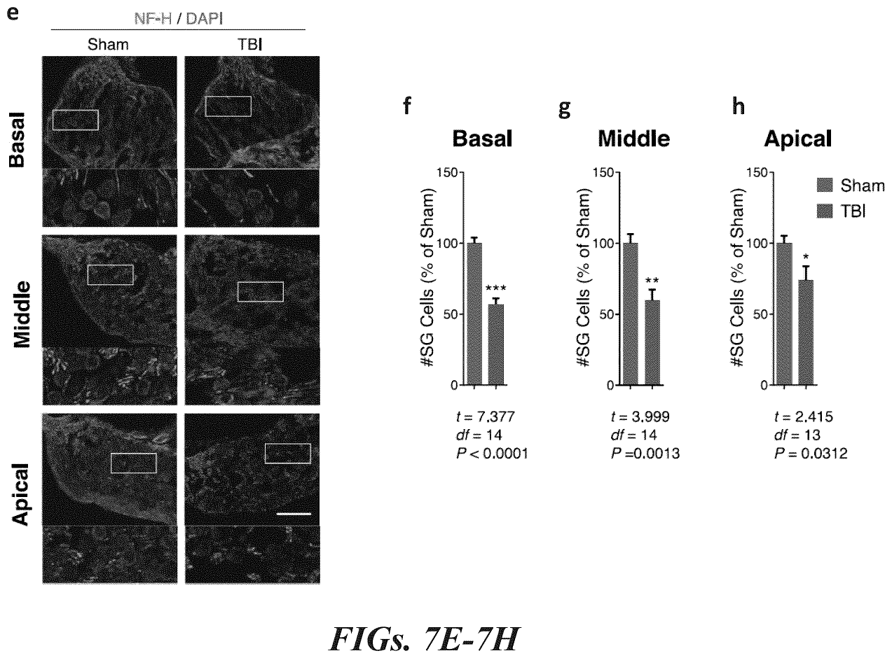

In order to determine cellular pathology that may be contributing to the hearing loss observed after rTBI, cochlear cryosections were stained with hematoxylin and eosin (H&E) and examined for structural differences (see FIGS. 7A-7H). A reduction in spiral ganglion cell (SGC) numbers were observed at 14, 30, and 60 dpi (see FIGS. 7B-7D). To further confirm whether rTBI induces loss of SGCs, a neuronal-specific marker, heavy chain neurofilament (NF-H), was used to selectively label SGCs at 14 dpi (see FIG. 7E). A significant reduction in NF-H positive SGCs in basal, middle, and apical regions of rTBI cochleae compared to sham was observed (see FIGS. 7F-7H).

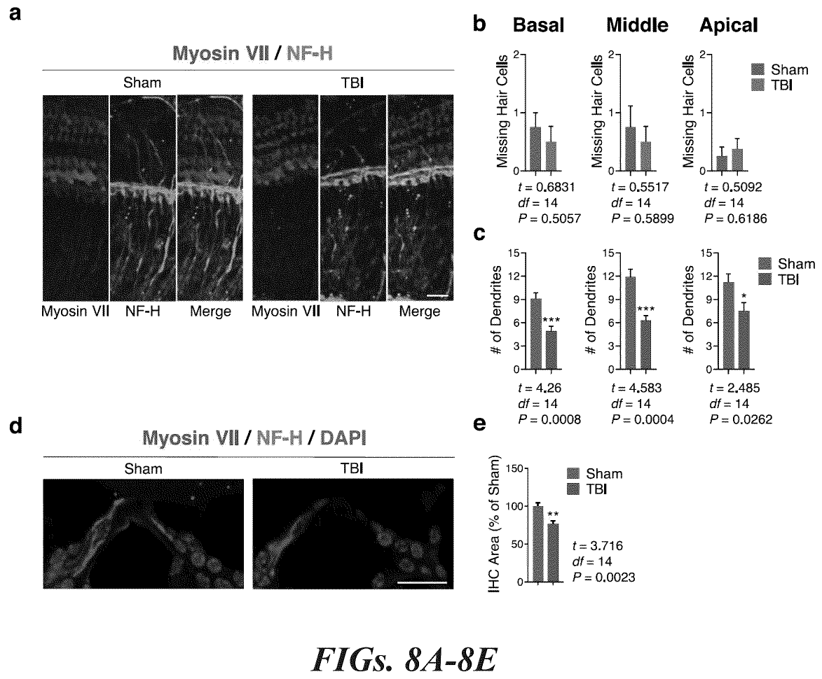

A small but significant effect of rTBI on ABR thresholds and P1 amplitude were observed at the earliest timepoint (14 dpi). This was accompanied by a loss of SGCs throughout the cochlea. In some cases, SGC loss may be secondary to the loss of hair cells, which promote SGC survival through the secretion of neurotrophic factors (Zhang et al., 2021). However, loss of SGC and synaptopathy have also been described in the absence of hair cell degeneration. In order to shed light on whether loss of hair cells contributed to rTBI-induced SGC loss, cochlear whole mounts of sham and rTBI mice at 14 dpi were prepared and stained for hair cells (Myosin VII) and neurites (NF-H) (see FIGS. 8A-8C). Few missing hair cells were observed in either sham or rTBI cochleae, and no significant difference in the number of missing hair cells could be detected at this time point (see FIG. 8B). In contrast, the number of spiral ganglion dendrites was significantly reduced in rTBI mice in all three regions of the cochlea (see FIG. 8C). This along with our staining data from cochlear cryosections suggests that loss of spiral ganglion occurs in the absence of any detectable change in hair cell number in the rTBI model, however, it does not preclude more subtle changes in hair-cell functionality, or hair cell degeneration at 30- and 60-dpi.

Loss of inner hair cell (IHC) volume has been linked to age-related hearing loss and may indicate a reduction in hair cell functionality (Liu et al., 2022). In order to explore changes in hair cell morphology after rTBI, cochlear cryosections were stained with Myosin VII, a commonly used hair cell marker (see FIGS. 8D-8E). A significant reduction in Myosin VII positive staining at 14 dpi was observed, which suggests deterioration of IHC morphology in the absence of overt hair cell loss.

Discussion The TBI mice exhibited neurodegeneration and neuroinflammation that persisted up to 60 dpi. This is in agreement with expected patterns of inflammation and cell death following TBI (Bodnar et al., 2019), which was documented in previous publications using this model (Das et al., 2019; Mayilsamy et al., 2020). Notably, behavioral changes—in the form of increased anxiety-like behaviors in an open field test, and increased anhedonia in a sucrose preference test-were observed following rTBI. These behaviors recapitulate common post-concussive symptoms seen in athletes and veterans with a history of repetitive rTBI (Albayram et al., 2020; Guskiewicz et al., 2007; Kerr et al., 2012; Stem et al., 2013). Major depression is the most commonly reported psychiatric diagnosis following TBI (Albayram et al., 2020; Fann et al., 2009). Evidence suggests that in patients with a history of TBI, depression is long-lasting and rates of depression increase over time (Jorge et al., 2004). This is in contrast to the observation that the effect of rTBI on anhedonia lessened over the course of two months. Although sucrose preference is only one measure of depression-like behavior in rodents, this represents a potential limitation to this model of rTBI.

A major finding of this study is that neurodegeneration in the auditory cortex was delayed compared to the injury site. This shows that the auditory cortex was not directly injured during TBI administration, but rather was affected by secondary injury spread. Although neurodegeneration had not spread to the auditory cortex at 14 dpi, hearing tests performed on sham and rTBI mice revealed increases in ABR thresholds beginning at 14 dpi and persisting throughout the testing period. At 14 dpi, ABR threshold shifts were minor and post-hoc analysis did not reveal any significant changes at individual frequencies. This may be partially due to greater variability in ABR thresholds in rTBI mice at this early timepoint. ABR threshold differences between sham and rTBI mice appeared to worsen over time. Differences were most apparent in the 6-20 kHz range, in which rTBI mice exhibited mean threshold shifts of approximately 10-15 dB at 30 dpi and 20-30 dB at 60 dpi.

Deficiencies in P1 amplitudes were also observed at 14 dpi. This is in agreement with our finding that spiral ganglion neuron numbers are reduced in rTBI mice at this timepoint. In contrast, no changes in P4 amplitude were observed after rTBI. This suggests that much of the damage is occurring near the beginning of the auditory pathway rather than in the brain or spinal column. In particular, reduced ABR P1 amplitudes indicate hidden hearing loss due to rTBI, accompanied by synapto-pathy. A deficient ABR GIN recovery ratio and increased MGT in these animals was observed. Reduced ability to process speech is a common complaint after TBI (Krause et al., 2014; Roup et al., 2020; Saunders et al., 2015; Turgeon et al., 2011). This ability is highly dependent upon auditory temporal resolution, measured here by worse GIN coding following rTBI (Dreschler and Leeuw, 1990; Osterhagen and Hildebrandt, 2018; Phillips, 1999; Saunders et al., 2015; Vijayasarathy et al., 2021).

Another major finding is that the number of spiral ganglion neurons in rTBI mice decreased at 14-60 dpi. Loss of SGCs at 14 dpi occurred in the absence of any detectable loss of hair cells. This is not the typical sequence of events in other forms of hearing loss such as induced by high intensity noise exposure. However, neurodegeneration in the absence of hair cell loss has been described in other contexts. For instance, moderate acoustic overexposure can cause temporary elevations in threshold that do not damage hair cells, but nonetheless lead to loss of synapses and spiral ganglion degeneration (Kujawa and Liberman, 2009; Shi et al., 2015). A similar pattern can be observed in aging humans and mice (Suzuka and Schuknecht, 1988; White et al., 2000). This type of cochlear neurodegeneration in the absence of hair cell loss is thought to underlie auditory processing issues such as speech intelligibility deficits, impaired hearing in noisy environments, auditory neuropathy, and tinnitus (Bao and Ohlemiller, 2010; Zhang et al., 2021). Importantly, many of the molecular changes that are suspected to contribute to age-related loss of spiral ganglion are also notable mechanisms of secondary injury following TBI. These include increased production of reactive oxygen species, inflammation, excitotoxicity, vascular changes, and others (Bao and Ohlemiller, 2010; Kociszewska and Vlajkovic, 202; Ladak et al., 2019; Ray et al., 2002; Seidman, 1998; Seidman et al., 2002). It is possible that these rTBI-induced changes in the brain and periphery can damage the auditory system in a similar but accelerated manner to that seen during normal aging.

One of the limitations of this study is that ABRs at high frequencies in sham and rTBI mice were not detected. This may be attributed to the use of used C57BL/6 mice, which have been shown to display a more rapid hearing loss in comparison to other mouse strains due to a single nucleotide variant in the gene that produces the stereocilia protein, cadherin 23 (Johnson et al., 1997, 2017). The pattern of hearing loss in these mice typically begins with losses in high frequencies as early as 1-2 months of age and progresses to lower frequencies over the next 12-15 months (Ison et al., 2007). This may explain the inability to detect ABRs at high frequencies in sham and rTBI mice over time. Due to extensive previous characterization and reports of rTBI in this mouse strain, we chose to continue using it despite its accelerated timeline of age-related hearing loss (also known as presbycusis). This limitation was controlled for by comparing rTBI mice to age-matched sham controls at all timepoints post-injury.

Another limitation of this study is the use of a male-only rTBI model of C57BL/6 mice that was previously characterized (Das et al., 2019; Mayilsamy et al., 2020). This is consistent with the observation that men exhibit a higher incidence of TBIs regardless of severity level. However, studies of TBI in rodents overwhelmingly neglect to include female mice; and seldom differentiate sex differences (Bodnar et al., 2019). Consideration of potential sex differences in TBI studies is an increasingly important topic as women's participation in contact sports and military service is on the rise (Biegon, 2021). Sex differences in hearing loss patterns in both humans and rodents have been well-documented (Lin et al., 2022; Milon et al., 2018; Reavis et al., 2023; Villavisanis et al., 2020). This is a limitation to our experiments that should be explored in future studies.

In this study, hearing loss in a closed-head CCI model of rTBI in C57BL6 male mice was demonstrated. The data indicate persistent changes in behavior, neurodegeneration, neuroinflammation, and hearing function up to 60 dpi. Spiral ganglion neuron loss at 14 dpi that occurred without loss of hair cells was observed, although changes in the morphology of inner hair cells and synapto-pathy could be detected. Hearing loss caused by rTBI may follow a similar retrograde sequence to that observed in age-related hearing loss or some cases of reversible noise-induced hearing loss. This model has the potential to be used in the exploration of mechanisms leading to auditory dysfunction after TBI, and the discovery of possible prevention and treatment strategies.

### Example 2: CCL20/CCR6 shRNA Mitigation Methods

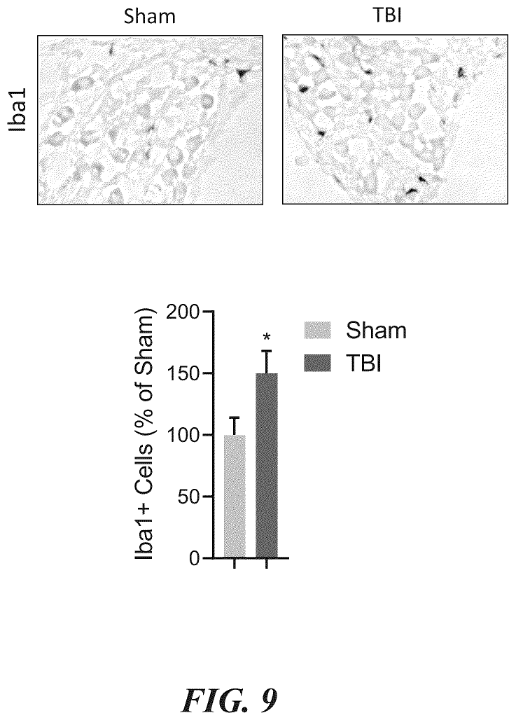

Referring now to FIG. 9, cochlear cryosections from 14 days post-injury (n=8) showed an increased number of Iba1+macrophages after rTBI, suggesting an inflammatory phenotype.

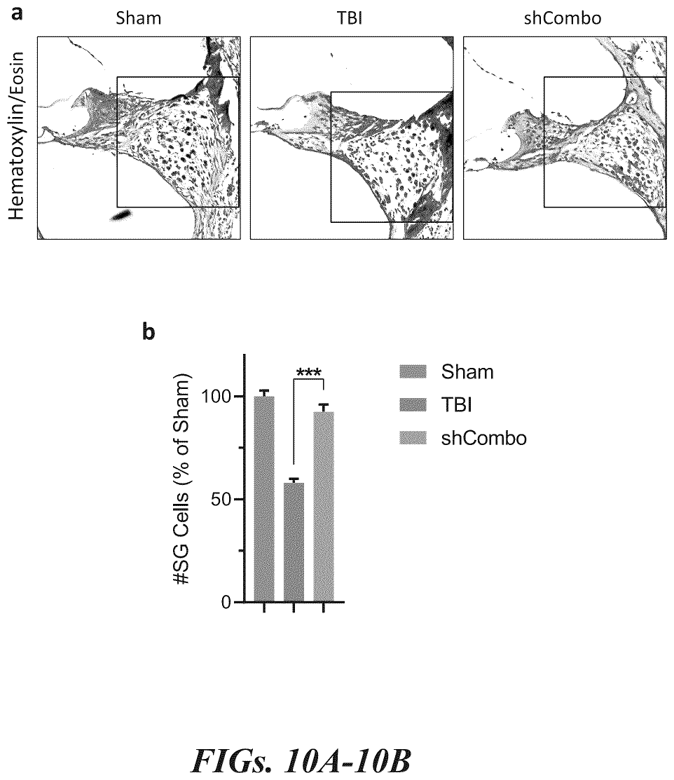

Referring now to FIGS. 10A and 10 B, FIG. 10A shows hematoxylin/eosin staining of cochlea from sham, rTBI, and rTBI mice treated with shRNA nanodendriplexes targeted to CCL20/CCR6 (shCombo) (n=3). It is interpreted that shCombo mice display less spiral ganglion neuron loss. FIG. 10B shows quantification of spiral ganglion cells from rTBI and shCombo mice (n=3).

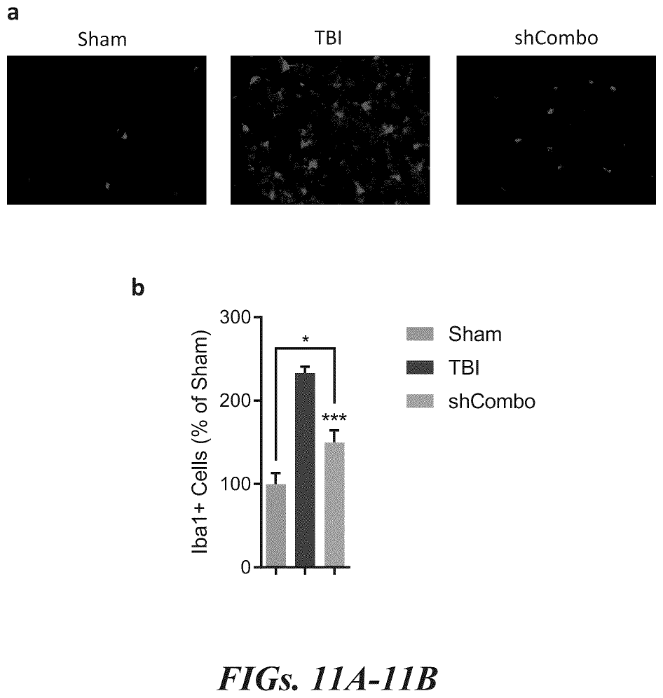

Referring now to FIGS. 11A and 11B, FIG. 11A shows Iba1 staining of auditory cortex from sham, rTBI, and shCombo mice (n=3). It is interpreted that shCombo mice display less Iba1+microglia. FIG. 11B shows quantification of Iba1+cells from rTBI, and shCombo mice (n=3).

### REFERENCES

Albayram, O., et al., 2020. Chronic traumatic encephalopathy—a blueprint for the bridge between neurological and psychiatric disorders. Transl. Psychiatry 10, 424. doi: 10.1038/s41398-020-01111-x.
Armstrong, R. C., et al., 2016. White matter involvement after TBI: clues to axon and myelin repair capacity. Exp. Neurol. 275, 328-333. doi: 10.1016/j.expneurol.2015.02.011, Pt 3.
Bao, J., Ohlemiller, K. K, 2010. Age-related loss of spiral ganglion neurons. Hear. Res. 264, 93-97. doi: 10.1016/j.heares.2009.10.009.
Barker-Collo, S., et al., 2015. Prevalence, natural course and predictors of depression 1 year following traumatic brain injury from a population-based study in New Zealand. Brain. Inj. 29, 859-865. doi: 10.3109/02699052.2015.1004759.
Biegon, A., 2021. Considering biological sex in traumatic brain injury. Front. Neurol. 12, 576366. doi: 10.3389/fneur.2021.576366.
Blennow, K., et al., 2016. Traumatic brain injuries. Nat. Rev. Dis. Primers 2, 16084. doi: 10.1038/nrdp.2016.84.
Bodnar, C. N., et al., 2019. A systematic review of closed head injury models of mild traumatic brain injury in mice and rats. J. Neurotrauma 36, 1683-1706. doi: 10.1089/neu.2018.6127.
Bombardier, C. H., et al., 2016. Depression trajectories during the first year after traumatic brain injury. J. Neurotrauma 33, 2115-2124. doi: 10.1089/neu.2015.4349.
Burda, J. E., et al., 2016. Astrocyte roles in traumatic brain injury. Exp. Neurol. 275, 305-315. doi: 10.1016/j.expneurol.2015.03.020, Pt 3.
Cha, J., et al., 2016. Abnormal hippocampal structure and function in clinical anxiety and comorbid depression. Hippocampus 26, 545-553. doi: 10.1002/hipo.22566.
Das, M., et al., 2019. CCL20-CCR6 axis modulated traumatic brain injury-induced visual pathologies. J. Neuroinflamm. 16, 115. doi: 10.1186/s12974-019-1499-z.
Donat, C. K., et al., 2017. Microglial activation in traumatic brain injury. Front. Aging Neurosci. 9, 208. doi: 10.3389/fnagi.2017.00208.
Dreschler, W. A., Leeuw, A. R, 1990. Speech reception in reverberation related to temporal resolution. J. Speech Hear. Res. 33, 181-187. doi: 10.1044/jshr.3301.181.
Fann, J. R., et al., 2009. Treatment for depression after traumatic brain injury: a systematic review. J. Neurotrauma 26, 2383-2402. doi: 10.1089/neu.2009.1091.
Faul, M., et al., 2010. Traumatic Brain Injury in the United States; Emergency Department Visits, Hospitalizations, and Deaths, 2002-2006. Centers for Disease Control and Prevention, National Center for Injury Prevention and Control, Atlanta (GA).
Gombay, N., Andrews, G. J., 2021. Living with embodied vibrations: sensory experiences following a traumatic brain injury. Soc. Sci. Med. 284, 114233. doi: 10.1016/j.socscimed.2021.114233.
Guskiewicz, K. M., et al., 2007. Recurrent concussion and risk of depression in retired professional football players. Med. Sci. Sports Exerc. 39, 903-909. doi: 10.1249/mss.0b013e3180383da5.
Heller, A.S., Bagot, R. C., 2020. Is hippocampal replay a mechanism for anxiety and depression? JAMA Psychiatry 77, 431-432. doi: 10.1001/jamapsychiatry.2019.4788.
Henry, J. A., et al., 2021. noise outcomes in servicemembers epidemiology (NOISE) study: design, methods, and baseline results. Ear. Hear. 42, 870-885. doi: 10.1097/AUD.0000000000000974.
Ishai, R., et al., 2018. Otopathologic changes in the cochlea following head injury without temporal bone fracture. Otolaryngol. Head Neck Surg. 159, 526-534. doi: 10.1177/0194599818769861.
Ison, J. R., et al., 2007. Age-related hearing loss in C57BL/6J mice has both frequency-specific and non-frequency-specific components that produce a hyperacusis-like exaggeration of the acoustic startle reflex. J. Assoc. Res. Otolaryngol. 8, 539-550. doi:10.1007/s10162-007-0098-3.
Johnson, K. R., et al., 1997. A major gene affecting age-related hearing loss in C57BL/6J mice. Hear. Res. 114, 83-92. doi:10.1016/s0378-5955(97)00155-x.
Johnson, K. R., et al., 2017. Effects of Cdh23 single nucleotide substitutions on age-related hearing loss in C57BL/6 and 129S1/Sv mice and comparisons with congenic strains. Sci. Rep. 7, 44450. doi:10.1038/srep44450.
Johnson, V. E., et al., 2013. Axonal pathology in traumatic brain injury. Exp. Neurol. 246, 35-43. doi:10.1016/j.expneurol.2012.01.013.
Jorge, R. E., et al., 2004. Major depression following traumatic brain injury. Arch. Gen. Psychiatry 61, 42-50. doi: 10.1001/archpsyc.61.1.42.
Karve, I. P., et al., 2016. The contribution of astrocytes and microglia to traumatic brain injury. Br. J. Pharmacol. 173, 692-702. doi: 10.1111/bph.13125.
Kaur, P., Sharma, S, 2018. Recent advances in pathophysiology of traumatic brain injury. Curr. Neuropharmacol. 16, 1224-1238. doi:10.2174/1570159X15666170613083606.
Kerr, Z. Y., et al., 2012. Nine-year risk of depression diagnosis increases with increasing self-reported concussions in retired professional football players. Am. J. Sports Med. 40, 2206-2212. doi: 10.1177/0363546512456193.
Kociszewska, D., Vlajkovic, S., 2022. Age-related hearing loss: the link between inflammaging, immunosenescence, and gut dysbiosis. Int. J. Mol. Sci. 23, 7348. doi:10.3390/ijms23137348.
Kozin, E. D., et al., 2021. Association of pediatric hearing loss and head injury in a population-based study. Otolaryngol. Head Neck Surg 165, 455-457. doi:10.1177/0194599820982904.
Krause, M. O., et al., 2014. Masking release, processing speed and listening effort in adults with traumatic brain injury. Brain. Inj. 28, 1473-1484. doi:10.3109/02699052.2014.920520.
Kujawa, S. G., Liberman, M. C., 2009. Adding insult to injury: cochlear nerve degeneration after “temporary” noise-induced hearing loss. J. Neurosci. 29, 14077-14085. doi:10.1523/JNEUROSCI.2845-09.2009.
Ladak, A. A., et al., 2019. A review of the molecular mechanisms of traumatic brain injury. World Neurosurg. 131, 126-132. doi:10.1016/j.wneu.2019.07.039.
Lamontagne, G., et al., 2022. Anxiety symptoms and disorders in the first year after sustaining mild traumatic brain injury. Rehabil. Psychol. 67, 90-99. doi:10.1037/rep0000422.
Lehman, E. J., et al., 2016. Suicide mortality among retired national football league players who played 5 or more seasons. Am. J. Sports Med. 44, 2486-2491. doi:10.1177/0363546516645093.
Lew, H. L., et al., 2011. Prevalence of dual sensory impairment and its association with traumatic brain injury and blast exposure in OEF/OIF veterans. J. Head Trauma Rehabil. 26, 489-496. doi:10.1097/HTR.0b013e318204e54b.
Lin, N., et al., 2022. Sex differences in the auditory functions of rodents. Hear. Res. 419, 108271. doi:10.1016/j.heares.2021.108271.
Lindquist, N. R., et al., 2020. Cochlear implantation after traumatic brain injury without otic capsule fracture: a case report and literature review. Am. J. Otolaryngol. 41, 102512. doi:10.1016/j.amjoto.2020.102512.
Liu, H., et al., 2022. Molecular and cytological profiling of biological aging of mouse cochlear inner and outer hair cells. Cell Rep. 39, 110665. doi:10.1016/j.celrep.2022.110665.
Liu, M. Y., et al., 2018. Sucrose preference test for measurement of stress-induced anhedonia in mice. Nat. Protoc. 13, 1686-1698. doi: 10.1038/s41596-018-0011-z.
Logue, M. W., et al., 2018. Smaller hippocampal volume in posttraumatic stress disorder: a multisite ENIGMA-PGC study: subcortical volumetry results from posttraumatic stress disorder consortia. Biol. Psychiatry 83, 244-253. doi:10.1016/j.biopsych.2017.09.006.
Mao, B., et al., 2021. Assessment of auditory and vestibular damage in a mouse model after single and triple blast exposures. Hear. Res. 407, 108292. doi:10.1016/j.heares.2021.108292.
Mayilsamy, K., et al., 2020. Treatment with shCCL20-CCR6 nanodendriplexes and human mesenchymal stem cell therapy improves pathology in mice with repeated traumatic brain injury. Nanomedicine 29, 102247. doi:10.1016/j.nano.2020.102247.
McClaskey, C. M., et al., 2020. A multi-metric approach to characterizing mouse peripheral auditory nerve function using the auditory brainstem response. J. Neurosci. Methods 346, 108937. doi:10.1016/j.jneumeth.2020.108937.
McKee, A. C., et al., 2015. The neuropathology of chronic traumatic encephalopathy. Brain Pathol. 25, 350-364. doi: 10.1111/bpa.12248.
Milon, B., et al., 2018. The impact of biological sex on the response to noise and otoprotective therapies against acoustic injury in mice. Biol. Sex Differ. 9, 12. doi:10.1186/s13293-018-0171-0.
Montgomery, S.C., Cox, B. C., 2016. Whole mount dissection and immunofluorescence of the adult mouse cochlea. J. Vis. Exp. e53561. doi:10.3791/53561.
Moreno-Lopez, L., et al., 2016. Depression following traumatic brain injury: a functional connectivity perspective. Brain Injury 30, 1319-1328. doi:10.1080/02699052.2016.1186839.
Moring, J. C., et al., 2018. Tinnitus, traumatic brain injury, and posttraumatic stress disorder in the military. Int. J. Behav. Med. 25, 312-321. doi:10.1007/s12529-017-9702-z.
Osterhagen, L., Hildebrandt, K. J, 2018. Ultra-fine temporal resolution in auditory processing is preserved in aged mice without peripheral hearing loss. bioRxiv, 363994 doi:10.1101/363994.
Paquette, S. T., et al., 2016. Noise exposure modulates cochlear inner hair cell ribbon volumes, correlating with changes in auditory measures in the FVB/nJ mouse. Sci. Rep. 6, 25056. doi:10.1038/srep25056.
Phillips, D. P., 1999. Auditory gap detection, perceptual channels, and temporal resolution in speech perception. J. Am. Acad. Audiol. 10, 343-354. doi:10.1055/s-0042-748505.
Popescu, C., et al., 2015. Actual data on epidemiological evolution and prevention endeavours regarding traumatic brain injury. J. Med. Life 8, 272.
Rattay, F., Danner, S. M., 2014. Peak I of the human auditory brainstem response results from the somatic regions of type I spiral ganglion cells: evidence from computer modeling. Hear. Res. 315, 67-79. doi:10.1016/j.heares.2014.07.001.
Ray, S. K., et al., 2002. Molecular mechanisms in the pathogenesis of traumatic brain injury. Histol. Histopathol. 17, 1137-1152. doi:10.14670/HH-17.1137.
Reavis, K. M., et al., 2023. Sex-linked biology and gender-related research is essential to advancing hearing health. Ear Hear. 44, 10-27. doi:10.1097/Aud.0000000000001291.
Roup, C. M., et al., 2020. Hearing Difficulties as a Result of Traumatic Brain Injury. J. Am. Acad. Audiol. 31, 137-146. doi:10.3766/jaaa.18084.
Saunders, G. H., et al., 2015. Auditory difficulties in blast-exposed veterans with clinically normal hearing. J. Rehabil. Res. Dev. 52, 343-360. doi:10.1682/JRRD.2014.11.0275.
Scholten, A. C., et al., 2016. Prevalence of and risk factors for anxiety and depressive disorders after traumatic brain injury: a systematic review. J. Neurotrauma 33, 1969-1994. doi:10.1089/neu.2015.4252.
Schumacher, A., et al., 2018. Ventral hippocampal CA1 and CA3 differentially mediate learned approach-avoidance conflict processing. Curr. Biol. 28, e1314. doi:10.1016/j.cub.2018.03.012.
Seibenhener, M. L., Wooten, M. C., 2015. Use of the open field maze to measure locomotor and anxiety-like behavior in mice. J. Vis. Exp. 52434. doi:10.3791/52434.
Seidman, M. D., 1998. Glutamate antagonists, steroids, and antioxidants as therapeutic options for hearing loss and tinnitus and the use of an inner ear drug delivery system. Int. Tinnitus J. 4, 148-154.
Seidman, M. D., et al., 2002. Molecular mechanisms of age-related hearing loss. Ageing Res. Rev. 1, 331-343. doi:10.1016/s1568-1637(02)00004-1.
Shangkuan, W. C., et al., 2017. Increased long-term risk of hearing loss in patients with traumatic brain injury: a nationwide population-based study. Laryngoscope 127, 2627-2635. doi:10.1002/lary.26567.
Shi, L., et al., 2015. Noise induced reversible changes of cochlear ribbon synapses contribute to temporary hearing loss in mice. Acta Otolaryngol. 135, 1093-1102. doi:10.3109/00 016489.2015.1061699.
Smith, K. D., et al., 2020. Hearing damage induced by blast overpressure at mild TBI level in a chinchilla model. Mil. Med. 185, 248-255. doi:10.1093/milmed/usz309.
Stern, R. A., et al., 2013. Clinical presentation of chronic traumatic encephalopathy. Neurology 81, 1122-1129. doi:10.1212/WNL.0b013e3182a55f7f.
Suzuka, Y., Schuknecht, H. F, 1988. Retrograde cochlear neuronal degeneration in human subjects. Acta Otolaryngol. Suppl. 450, 1-20. doi:10.3109/00016488809098973.
Swan, A. A., et al., 2018. Sensory dysfunction and traumatic brain injury severity among deployed post-9/11 veterans: a chronic effects of neurotrauma consortium study. Brain Injury 32, 1197-1207. doi:10.1080/02699052.2018.1495340.
Terpstra, A. R., et al., 2017. Higher Anxiety symptoms predict progressive hippocampal atrophy in the chronic stages of moderate to severe traumatic brain injury. Neurorehabil. Neural Repair 31, 1063-1071. doi:10.1177/1545968317736817.
Turgeon, C., et al., 2011. Auditory processing after sport-related concussions. Ear Hear. 32, 667-670. doi:10.1097/AUD.0b013e31821209d6.
Uchiyama, M., et al., 2021. Temporal bone pathology secondary to head trauma—a human temporal bone study. Otol. Neurotol. 42, e1152-e1159. doi:10.1097/MAO.0000000000003192.
Vijayasarathy, S., et al., 2021. Speech perception in noise, gap detection and amplitude modulation detection in suspected hidden hearing loss. Hear. Balanc. Commun. 19, 203-211. doi:10.1080/21695717.2021.1876494.
Villavisanis, D. F., et al., 2020. Sex-based differences in hearing loss: perspectives from non-clinical research to clinical outcomes. Otol. Neurotol. 41, 290. doi:10.1097/MAO.0000000000002507.
Webner, D., Iverson, G. L., 2016. Suicide in professional American football layers in the past 95 years. Brain Injury 30, 1718-1721. doi:10.1080/02699052.2016.1202451.
White, J. A., et al., 2000. Pattern of degeneration of the spiral ganglion cell and its processes in the C57BL/6J mouse. Hear Res. 141, 12-18. doi:10.1016/s0378-5955(99)00204-x.
Williamson, T. T., et al., 2015. Auditory brainstem gap responses start to decline in mice in middle age: a novel physiological biomarker for age-related hearing loss. Cell Tissue Res. 361, 359-369. doi:10.1007/s00441-014-2003-9.
Zhang, L., et al., 2021. Mechanism and prevention of spiral ganglion neuron degeneration in the cochlea. Front. Cell. Neurosci. 15, 814891. doi:10.3389/fncel.2021.814891.

What is claimed is:

1. A method of treatment for auditory dysfunction, the method comprising:
administering a therapeutic amount of a therapeutic agent to inhibit chemokine ligand 20 (CCL20) and/or chemokine receptor 6 (CCR6) one of a dendrimer nanoparticle complexed with at least one short hairpin RNA (shRNA)-encoding DNA plasmid of at least one dendriplex, or a PPARγ agonist; and
administering a therapeutic amount of stem cells.

2. The method of claim 1, wherein auditory dysfunction is a result of traumatic brain injury.

3. The method of claim 1, wherein the therapeutic agent to for down-regulating CCL20 and/or CCR6 comprises one of a dendrimer nanoparticle complexed with at least one short hairpin RNA (shRNA)-encoding DNA plasmid of at least one dendriplex, or a peroxisome proliferator-activated receptor gamma (PPARγ) agonist.

4. The method of claim 3, wherein the at least one shRNA-encoding DNA plasmid contains at least one shRNA encoding CCL20, CCR6 or a combination thereof.

5. The method of claim 3, wherein the PPARγ agonist comprises pioglitazone rosiglitazone, troglitazone, englitazone, balaglitazone, rivoglitazone, ciglitazone, lobeglitazone, or netoglitazone, honokiol, amorfrutin 1, amorfrutin B, and amorphastilbol, or a pharmaceutically acceptable salt thereof.

6. The method of claim 5, wherein the PPARγ agonist comprises two or more PPARγ agonists.

7. The method of claim 1, wherein the or a pharmaceutically acceptable salt thereof.

8. The method of claim 1, wherein the stem cells are human mesenchymal stem cells.

9. The method of claim 1, wherein the therapeutic agent to for down-regulating chemokine ligand 20 (CCL20) and/or chemokine receptor 6 (CCR6) is administered before administering the stem cells.

10. The method of claim 1, wherein the therapeutic agent to down-regulating chemokine ligand 20 (CCL20) and/or chemokine receptor 6 (CCR6) is administered concurrently with administering the stem cells.

11. The method of claim 1, wherein the therapeutic agent to for down-regulating chemokine ligand 20 (CCL20) and/or chemokine receptor 6 (CCR6) is administered after administering the stem cells.

12. The method of claim 1, wherein the therapeutic agent to for down-regulating chemokine ligand 20 (CCL20) and/or chemokine receptor 6 (CCR6) is administered intranasally.

13. The method of claim 1, wherein the therapeutic agent to for down-regulating chemokine ligand 20 (CCL20) and/or chemokine receptor 6 (CCR6) is administered orally.

14. The method of claim 1, wherein the therapeutic agent to for down-regulating chemokine ligand 20 (CCL20) and/or chemokine receptor 6 (CCR6) is administered intravenously.

15. The method of claim 1, wherein the stem cells are administered intravenously.

16. The method of claim 1, wherein the stem cells are administered intranasally.

17. The method of claim 1, wherein the stem cells are administered intra-arterially.

18. The method of claim 1, wherein the stem cells are administered intra-cranially.
